(19) United States
(12) Patent Application Publication
(10) Pub. No.: US 2020/0071774 A1
(43) Pub. Date: Mar. 5, 2020

(54) Systems and Methods for Securing Anti-Tamper Label and Tape Adhesive Chemistry with Molecular Taggants

(71) Applicant: Invisidex, Inc., Ojai, CA (US)

(72) Inventors: Reep Paul, Ojai, CA (US); Kabir Sagoo, Ojai, CA (US)

(21) Appl. No.: 16/557,565

(22) Filed: Aug. 30, 2019

Related U.S. Application Data
(60) Provisional application No. 62/726,175, filed on Aug. 31, 2018.

(51) Int. Cl. C12Q 1/6888; C12Q 1/6876; G16B 50/00

(52) U.S. Cl. CPC C12Q 1/6888; G16B 50/00; C12Q 1/6876

(57) ABSTRACT

The present invention describes systems and methods for formulating anti-tamper label and security tape adhesive chemistry with nucleic acid identifier molecular markers to authenticate the tape or label affixed to an article.

### CROSS-REFERENCED APPLICATIONS

This application claims priority to U.S. Provisional Patent Application No. 62/726,175 which was filed Aug. 31, 2018.

### FIELD OF THE INVENTION

The present invention relates to the fields of chemistry, biochemistry, material science, analytical tools, microbiology, and adhesives. The present invention also relates to supply chain management, authentication, anti-counterfeiting, quality control, tamper deterrence, product traceability, and computer networks.

### BACKGROUND

Frangible labels, security tapes, and security labels are routinely used on articles such as products or product packaging for authentication and supply chain traceability purposes. These frangible labels, security tapes and labels may have special security features such as holograms, serialization, or other means of authenticating that label affixed to an article.

Security tapes, labels, and frangible labels are routinely affixed on articles in aircraft, cargo container, consumer electronics, shipping, automotive, manufacturing and other industries. Various tapes and labels have different mechanisms for preventing, deterring or identifying tampering of the label. For example only, one such label tears easily and reveals a hidden layer if improperly removed from a product surface. Other tapes have features such as printed logos, writing, and dual layered messages.

### DESCRIPTION OF DRAWINGS

Figure depicts a label peeled back to reveal residual adhesive on the article surface; and

FIG. 2 depicts a label with adhesive applied.

### SUMMARY OF THE INVENTION

In various embodiments, the invention relates to analyzing data associated with taggants contained within an adhesive material for authenticating the provenance of a tape or label present on an article. In particular the present invention relates to embedding identification data into an adhesive material that is present on anti-tamper labels or tape affixed to certain articles, as well as the presence of identification data in the residue left behind by the label or tape.

Anti-tamper tape and label technologies are subject to forgery and counterfeiting if they lack covert security features or use visual security systems such as holograms or pigments and dyes. Furthermore, the tape or label can be compromised. If it does not leave a residue on the product surface, it may be difficult to confirm the tape has not been moved.

Physical and chemical taggants were developed to protect at-risk materials such as fuels, bulk chemicals, and other products. These taggants can be composed of organic molecules, inorganic fluorescent markers, or nucleic acids such as deoxyribonucleic acid (DNA). Conventional taggant technologies contain data about products or they are associated with authentication and traceability data. If the taggant does not contain data that can be interrogated and decrypted, its mere presence alone may indicate an article's authenticity.

DNA authentication technologies are gaining traction in various industries because of DNA's scalability as well as its ability to hide in plain site; DNA can be embedded in materials without disclosing the exact DNA sequence. Like barcodes in the super market or unique fingerprints, DNA sequences are data codes that can be customized for a unique authentication signature.

DNA sequences are synthesized into physical DNA molecules. The synthesized DNA molecules can be embedded into materials such chemicals or surface witness marks for authentication purposes. Due to the prevalence of DNA in environmental conditions, unless a user knows the target sequence they are looking for, it is nearly impossible to interrogate a DNA taggant using the polymerase chain reaction (PCR) analytical method.

Anti-tamper security tapes and labels as well as other frangible labels can be forged or counterfeited; therefore, there is a critical need to authenticate security tapes and labels with molecular identifier taggants blended into their adhesive chemistry.

The present invention enables a new marking system for anti-tamper tapes and security labels as well as articles containing residue of those tapes and labels enabled by molecular marker identifier taggants.

The present invention allows for the interrogation of taggant data embedded within an adhesive material present on a security tape or label affixed to an article as well as the residue left behind after removing the tape or label from an article. The present invention allows for the interrogation of taggant data embedded within an adhesive material present on a security tape or label affixed to an article as well as the residue left behind after removing the tape or label from an article.

Various anti-tamper labels, security tapes and frangible labels may be designed to leave a residue if the label is removed from an article's surface, or, they may be residue-less. In either method, the label contains adhesive chemistry that encourages the affixing of the label to a surface.

Anti-tamper labels can be removed, mimicked, defeated, or counterfeited. Therefore, there is a critical need for labels and security tapes with unique, covert identifiable information stored in the adhesive chemistry to uniquely authenticate the tape.

In one embodiment of the present invention, molecular markers and identifier taggants are blended into the adhesive chemistry used to bond the label to a surface. This method increases the likelihood that an article was labeled with the correct security tape because unauthorized personnel may attempt to remove the security tape, modify the article, and then replace the tape or place a counterfeit label. If the corresponding molecular identifier data is not present on the label but is present on the residue, it is possible to determine someone altered the tape.

In another embodiment of the present invention, the security label or tape may be a frangible label or may be structured to contain layers that can be peeled or exposed by design. The covert molecular markers containing identifier data may be present in any layer or film within these intermediate label surfaces.

In another embodiment of the present invention, the adhesive chemistry contains embedded nucleic acids such as deoxyribonucleic acid (DNA) or ribonucleic acid (RNA). The DNA taggants are introduced to the adhesive chemistry formulation during the adhesive formulation process to ensure homogeneous distribution prior to applying the adhesive on the label.

### DETAILED DESCRIPTION OF THE PREFERRED EMBODIMENTS

DNA taggants are short nucleic acid fragment data sequences that can be associated with a lookup table with anti-tamper tape and security label identification data. The data contained in DNA taggants can be read using a polymerase chain reaction (PCR) instrument or sequencing device.

Various anti-tamper labels, security tapes and frangible labels may be designed to leave a residue if the label is removed from an article's surface, or, they may be residue-less. In either method, the label contains adhesive chemistry that encourages the affixing of the label to a surface.

Anti-tamper labels can be removed, mimicked, defeated, or counterfeited. Therefore, there is a critical need for labels and security tapes with unique, covert identifiable information stored in the adhesive chemistry to uniquely authenticate the tape.

In one embodiment of the present invention shown in FIG. 1, molecular markers 100 and identifier taggants 110 are blended into the adhesive chemistry used to bond a label 120 to a surface or article. This method increases the likelihood that an article was labeled with the correct security tape because unauthorized personnel may attempt to remove the security tape, modify the article, and then replace the tape with a counterfeit label. If the corresponding molecular identifier data is not present on the label, but is present on the residue, it is possible to determine someone altered the tape.

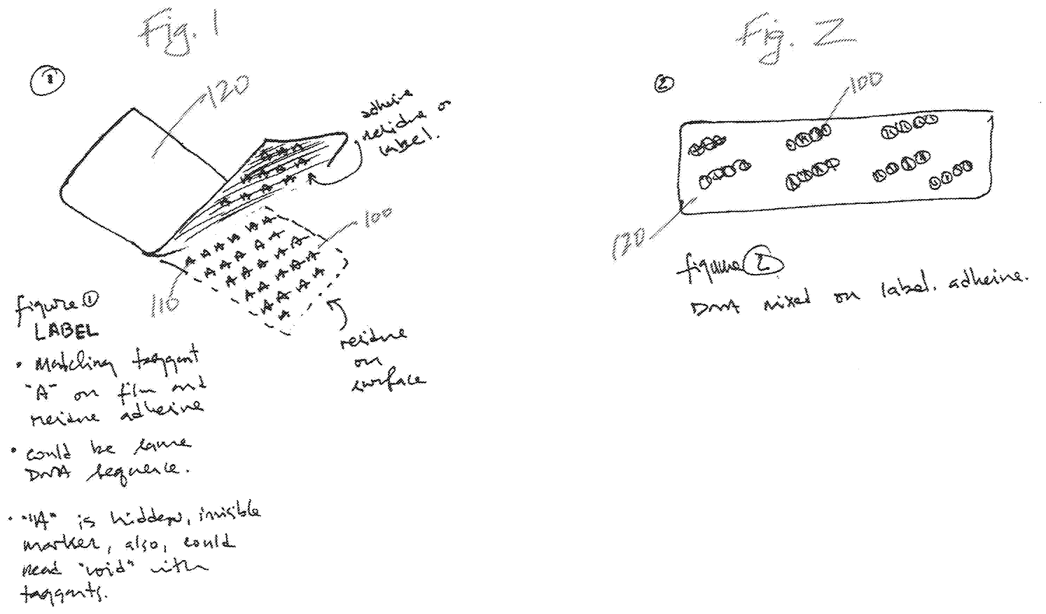

In another embodiment of the present invention shown in FIG. 2, the security label or tape 120 may be a frangible label or may be structured to contain layers that can be peeled or exposed by design. The covert molecular markers containing identifier data may be present in any layer or film within these intermediate label surfaces.

In another embodiment of the present invention, the adhesive chemistry contains embedded nucleic acids such as deoxyribonucleic acid (DNA) or ribonucleic acid (RNA). The DNA taggants are introduced to the adhesive chemistry formulation during the adhesive formulation process to ensure homogeneous distribution prior to applying the adhesive on the label.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters-Patent is:

1. An authentication security protection system that confirms an original and correct anti-tamper security label or tape is affixed to a corresponding article, comprising:
an adhesive containing molecular markers; and
a tape having the adhesive on the surface applied against an article.

2. A system of claim 1, where the molecular markers are a nucleic acid.

3. A system of claim 2, where the nucleic acid is deoxyribonucleic acid (DNA).

4. A system of claim 2, where the nucleic acid is ribonucleic acid (RNA).

5. An authentication security protection system for confirming the presence of an original, correct security label or tap affixed to an article, the system comprising:
a security label having layers; and
an adhesive containing molecular markers, the adhesive being placed on a surface of the security label or tape applied against an article.

6. A system as set for in claim 5, wherein adhesive containing molecular markers is placed between the layers of the security label or tape.

7. A system as set forth in claim 6, wherein the adhesive containing molecular markers applied to the surface between the security label or tape, is different from the adhesive containing molecular markers that is applied between the layers of the security label or tape.

8. An authentication security system for confirming the presence of an original correct security label or tape affixed to an article, the system comprising:
a first layer of film affixed to an article with an adhesive; and
a second layer of film affixed to the first layer of film by an adhesive containing molecular markers.

9. An authentication security protection system as recited in claim 1, wherein the molecular markers are a nucleic acid.

10. An authentication security protection system as recited in claim 1, wherein the nucleic acid is deoxyribonucleic acid (DNA).

11. An authentication security protection system as recited in claim 1, wherein the nucleic acid is ribonucleic acid (RNA).

12. A method for authenticating an anti-tamper security label affixed to an article, the method comprising the steps of:
formulating an adhesive containing molecular markers having a predetermined DNA sequence;
applying the adhesive to a surface of a label;
applying the label to an article such that the label will need to be removed to open or gain access to the article; and
removing the label and using polymerase chain reaction analysis to verify that the residual adhesive on the label has the same DNA sequence molecular markers as in the original adhesive.
